(19) United States
(12) Patent Application Publication
(10) Pub. No.: US 2016/0242527 A1
(43) Pub. Date: Aug. 25, 2016

(54) AROMATIC DIFFUSER APPLICABLE TO COVERS OR CASES FOR ELECTRONIC DEVICES, WALLETS AND OTHER WEARING ACCESORIES

(71) Applicant: FRANCISCO JAVIER LOPEZ RIVERIEGO, Ubrique (ES)

(72) Inventor: FRANCISCO JAVIER LOPEZ RIVERIEGO, Ubrique (ES)

(21) Appl. No.: 14/626,036

(22) Filed: Feb. 19, 2015

(51) Int. Cl. A45D 34/00; A61L 9/12; A01M 29/12

(52) U.S. Cl. CPC A45D 34/00; A01M 29/12; A61L 9/12; A45D 2034/007

(57) ABSTRACT

Scent diffuser for electronic device cases, purses and other personal complements. It's made of a light mesh, a thin absorbent piece and a waterproof plastic sheet. It diffuses essence fragrances.

### TECHNICAL FIELD OF THE INVENTION

The proposed invention belongs to the field of covers or cases for mobile electronic devices such as phones, PDAs, tablets, laptops, cameras, etc. and it is also useful for handbags, wallets and other wearing accesories.

### BACKGROUND OF THE INVENTION

There are several electronic devices in the market that make our lives easier with a wide range of uses, which we protect with cases or covers. Cases have also a strong esthetic component, allowing us to match our devices with our tastes or our personality. Nowadays, they are part of the dressing code, in the same way we could think about an outfit.

However, those cases are no more than an envelope which we use to decorate our devices, in the same way as we do with our wallets or handbags. In this accesories, the value is in fact in the inside, but we can not forget that their esthetic component and configuration make a strong effect on us, because they are accesories always visible and that we use to represent our style. In this sense, a mobile phone case could resemble a perfume; a liquid that covers a person with a given scent, with no more presumptions.

In addition, a perfume is often incompatible with an aromatic solution used for allergic or respiratory problems, and even with insect repellent; due to this, the only way is to mix different fragances with the expected and undesired result, or refuse to the benefits of one product instead of the other.

As we can see, we have several resources in our hands but they cannot be used at the same time always. There are people that find a solution to their illness in aromatherapy, but they should use a pendant or another object coated in the perfume. Others would need to use ultrasonic devices as insect repellents, in order to avoid the mixing of fragances or due to be allergic to some products of the repellents.

In other cases, returning to our invention—the covers—, we find that the confidence in the choice that consumer from our environment performs lead us to repeat it to the point of copying even the cover, what may cause the mistake of mixing up the devices.

This would occur less, for example, in the case of handbags.

### BRIEF DESCRIPTION OF THE INVENTION

Given that the different covers of mobile devices (telephones, cameras, tablets, laptops, etc.) and wearing accesories (handbags, wallets, etc.) are not easy to distinguish when they match up other similar products—they are not a benefit for us in spite of being constantly by our side or in our hands or pockets, and also that perfumes or fragances are not easily combinable between them, impeding that some people could enjoy the benefits of aromatherapy, or letting them only if they refuse to use their personal perfume, or making them refuse to use some insect repellent because of being uncompatible with perfumes, the present invention manage to join both resources, turning a mobile phone cover in the ideal frame of an aromatic diffuser that will allow not only to distinguish the cover of another similar due to the smell, but also to facilitate therapeutic benefits of aromatherapy or even repell insects without the need of use different devices or elements, nor mixing fragances or stop using ones instead of others.

Any of this elements—a mobile phone cover for instance, will show in the folding piece a small window—without disrupting the final shape or size of the final product, that will have behind it a group of elements including a thin mail that contains and leave visible a thin piece made of a proper material—wood, cellulose, etc., that allows the cover to be coated with perfume through the mail, all the times we want and with a slow releasing of the aromatic liquids.

The grup of elements contains, finally, a thin plastic layer with the purpose of avoiding that the liquids stain the cover and the device. This layer is not visible once it is sewed to the cover.

When the invention is used in handbags, it could be placed in the handles of the handbag, for instance, because this invention is adaptable to any shape required and is also pretty flexible.

### DESCRIPTION OF THE DRAWINGS

In order to compliment the given description, and with the purpose of a better understanding of the characteristics of this invention, in agreement with a preferred example of development of this invention, it is given a drawing where, with illustrative purpose and non limitative, have been represented the following:

FIG. 1—In this, it can be seen the window (4) made in the stiff base of the folding tap and also in the leather piece that covers it and that creates, sticked by its back side, the mail (1).

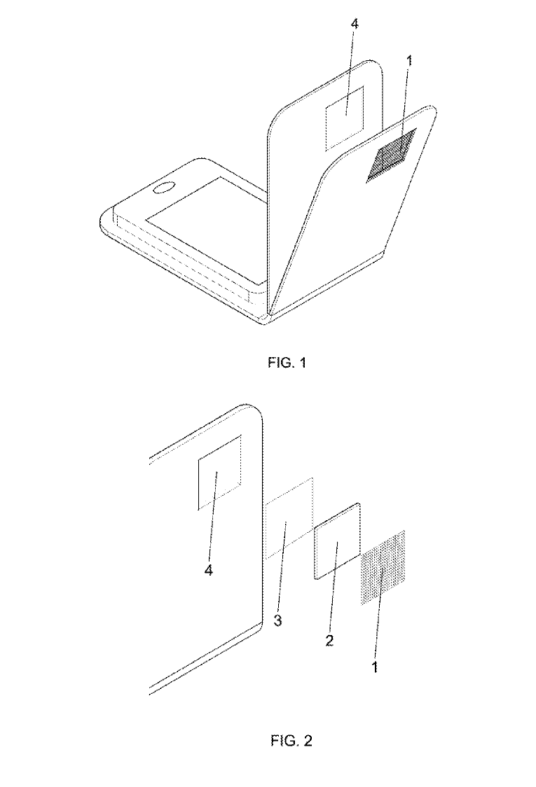

FIG. 2—In this view is shown in an ordered way the different elements of the invention:
1. Pierced mail
2. Coatable piece, porous and flexible.
3. Insulating plastic
4. Window

### PREFERRED REALIZATION OF THE INVENTION

Starting from a slot as a window made in covers or handles, it is placed behind the leather and centered in that window a diffuser system that includes a pierced mail (1); behind it is placed a proper coatable porous and flexible piece (2) through the piercings of the mail (1), and behind it is placed a thin plastic layer (3) that avoids the liquids to pass through.

The diffusing system is hidden behind the cover; it can be refilled every time we want by its back side, through the piercings of the mail (1).

1. Aromatic diffuser applicable to electronic devices covers, handbags and other personal accesories, characterized for including a window (4) upon the folding base of the cover or handle of the complement, that have behind it the diffusing system, made by a mail (1) that holds a coatable, porous and flexible piece (2) and a thin plastic layer (3) that avoids the liquids to pass through; all this is sticked and hidden to the sigh behind the cover.

2. Aromatic diffuser applicable to electronic devices covers, handbags and other personal accesories characterized for its coatable piece, which can be refilled though the piercings of the mail, that are also used to release the fragances slowly.

3. Aromatic diffuser applicable to electronic devices covers, handbags and other personal accesories characterized because, by the combination of personal fragances which coates the porous piece (2), two apparently identic covers can be easily distinguished.
